(19) United States
(12) Patent Application Publication
(10) Pub. No.: US 2008/0287649 A1
(43) Pub. Date: Nov. 20, 2008

(54) Methods for the synthesis of cyclic peptides

(76) Inventors: Lin Chen, Superior, CO (US); Yeun-Kwei Han, Louisville, CO (US); Christopher R. Roberts, Berthoud, CO (US)

Correspondence Address: ROCHE PALO ALTO LLC;Patent Law Department, M/S A2-250, 3431 Hillview Avenue, Palo Alto, CA, 94304, US

(21) Appl. No.: 12/004,265

(22) Filed: Dec. 20, 2007

Related U.S. Application Data
(60) Provisional application No. 60/877,780, filed on Dec. 29, 2006.

(51) Int. Cl. C07K 2/00; C07C 69/616; C07K 5/12

(52) U.S. Cl. 530/317; 560/39

(57) ABSTRACT

Methods for the synthesis of cyclic peptides are provided, as well as novel dipeptide compounds. The methods include the solid phase synthesis of a dipeptide, which is the coupled to a second peptide in a solid phase reaction. The peptide is then cyclized following the coupling reaction. The methods and dipeptides are particularly useful for the synthesis of MC-4 receptor agonist peptides.

### PRIORITY CLAIM

The present non-provisional patent application claims benefit from United States Provisional Patent Application having Ser. No. 60/877,780, filed on Dec. 29, 2006, by Roberts et al., and titled METHODS FOR THE SYNTHESIS OF CYCLIC PEPTIDES, wherein the entirety of said provisional patent application is incorporated herein by reference.

### FIELD OF THE INVENTION

The invention relates to the synthesis of cyclic peptides.

### BACKGROUND OF THE INVENTION

Many methods for peptide synthesis are described in the literature (for examples, see U.S. Pat. No. 6,015,881; Mergler et al. (1988) Tetrahedron Letters 29:4005-4008; Mergler et al. (1988) Tetrahedron Letters 29:4009-4012; Kamber et al. (eds), Peptides, Chemistry and Biology, ESCOM, Leiden (1992) 525-526; Riniker et al. (1993) Tetrahedron Letters 49:9307-9320; Lloyd-Williams et al. (1993) Tetrahedron Letters 49:11065-11133; Andersson et al. (2000) Biopolymers 55:227-250; and Bray, B. L. (2003) Nature Reviews 2:587-593. The various methods of synthesis are distinguished by the physical state of the phase in which the synthesis takes place, namely liquid phase or solid phase.

In some cases, the synthesis process includes reactions that provide a cyclic peptide. Peptides can be cyclized to rigidify their structure and improve their in vivo stability (see Camarero and Muir, (1999) J. Am. Chem. Soc., 121:5597 5598). Cyclic peptides can be more difficult to degrade by body enzymes and can also have increased affinity to receptors in the body.

Various synthesis processes for the preparation of cyclic peptides have been described in the prior art. In some cases these involve the cyclization of a linear polypeptide. For example, a chemical cross-linking approach was used to prepare a backbone cyclized version of bovine pancreatic trypsin inhibitor (Goldenburg and Creighton (1983) J. Mol. Biol., 165:407 413). Other approaches include chemical (Camarero, et al., (1998) Angew. Chem. Int. Ed., 37:347 349; Tam and Lu (1998) Prot. Sci., 7:1583 1592; Camarero and Muir (1997) Chem. Commun., 1997:1369 1370; and Zhang and Tam (1997) J. Am. Chem. Soc. 119:2363 2370) and enzymatic (Jackson et al., (1995) J. Am. Chem. Soc., 117:819 820) intramolecular ligation methods which allow linear synthetic peptides to be efficiently cyclized under aqueous conditions.

The synthesis of non-recombinant cyclic peptides typically involves solid or solution phase chemical synthesis steps. In these synthesis steps, amino acids or peptides with protecting groups are generally used as they have reactive side groups as well as reactive terminal ends. Undesired reactions at side groups or at the wrong terminal end of a reactant can produce undesirable by-products, sometimes in significant quantities. These by-products and reactions can seriously impair yield or even ruin the product being synthesized from a practical perspective. To minimize side reactions, it is conventional practice to appropriately mask reactive side groups and (alpha amino) terminal ends of reactants to help ensure that the desired reaction occurs.

The use of Fmoc (fluorenylmethyloxycarbonyl) chemistry for protection of the alpha amino group has become the preferred method for most contemporary solid and solution phase peptide synthetic processes. Fmoc chemistry has also been shown to be more reliable and produce higher quality peptides than Boc (t-butoxycarbonyl) chemistry. In Fmoc synthesis, removal of the Fmoc protecting group to provide a reactive amino terminus is typically performed in the presence of a mild base, such as piperidine. After base treatment, the nascent peptide is typically washed and then a mixture including an activated amino acid and coupling co-reagents is placed in contact with the nascent peptide to couple the next amino acid. After coupling, non-coupled reagents can be washed away and then the protecting group on the N-terminus of the nascent peptide can be removed, allowing additional amino acids or peptide material to be added to the nascent peptide in a similar fashion.

In Fmoc chemistry, reactive side chain groups of the amino acid and peptide reactants, including the resin-bound peptide material as well as the additional material to be added to the growing chain, typically remain masked with side chain protecting groups throughout synthesis. Generally, side chain protecting groups are used that are not removed during deprotection of the alpha amino protecting group (i.e., piperidine treatment) during synthesis. Commonly used side chain protecting groups in Fmoc chemistry are removable by acidolysis (e.g., using TFA) and include Acm (acetamidomethyl), Boc, Mtr (methoxytrimethylbenzenesulphonyl), OtBu (t-butyl ester), Pbf (2,2,4,6,7-pentamethyl-dihydrobenzofuran-5-sulfonyl), Pmc (2,2,5,7,8-pentamethylchroman-6-sulphonyl), tBu (t-butyl), and Trt (trityl). In Fmoc chemistry, these protecting groups are available on certain amino acids, as permitted by the chemical structure of the side chain.

While there is current widespread use of solid phase chemistries such as Fmoc, there are circumstances wherein using these chemistries can be problematic. For example, in some cases, after Fmoc removal, the peptide intermediate may suffer from an undesired side reaction leading to a dead-end product. In other cases, steps of the synthesis process may demonstrate low and unacceptable reproducibility.

These problems can be exacerbated in cyclic peptide synthesis processes, which typically require difficult coupling reactions to promote coupling between selected amino acid side chains.

The present invention addresses these problems and provides advances and improvements in the art of synthesizing cyclic peptides.

### SUMMARY OF THE INVENTION

The present invention provides novel methods for the production of cyclic peptides. The present invention also provides novel peptide compounds that include an aspartic acid residue and a non-natural amino acid. These peptide compounds can be used as intermediates for the synthesis of cyclic peptides, such as cyclic melanocortin-4 receptor agonist peptides.

In one aspect, the invention provides a particularly effective and efficient method for the preparation of cyclic peptides. The method provides routes for overcoming difficulties in the synthesis of cyclic peptides, such as the formation of dead-end intermediate peptides. The methods advantageously provide processing benefits associated with the production of cyclic peptides, such as a reduction of the amount of reagents or elimination of certain processing techniques typically used in the synthesis of cyclic peptides. Overall, the methods of the invention provide a higher production yield of the cyclic peptide product and improved cyclic peptide purity.

Generally, the method of the present invention involves the solid phase synthesis of at least two peptide intermediate fragments, one of which is a dipeptide. The process avoids formation of an dead-end intermediate fragment, which may otherwise be formed if the dipeptide approach is not used.

According to the invention a method for forming a cyclic peptide is provided. The method comprises the following steps. First, a dipeptide fragment is synthesized on a resin, wherein the synthesis provides a dipeptide fragment comprising an amino acid residue with a first side chain. The dipeptide is then cleaved from the resin. A second peptide fragment coupled to a resin is provided, the second peptide comprising an amino acid residue with a second side chain. The carboxyl terminus of the cleaved dipeptide fragment is then coupled to the amino terminus of the second peptide fragment, thereby forming a third peptide, which is resin bound. The method also comprises a step of cyclizing the third peptide by covalently coupling the first side chain of the dipeptide portion with the second side chain of the second peptide portion.

In some cases, the step of cyclizing comprises coupling an acidic (first) side chain to a basic (second) side chain. As an example, the peptide is cyclized via the side chain of an aspartic acid residue and the side chain of a lysine residue.

This method can be used for the synthesis of cyclic peptides that include non-natural amino acids. Such non-natural amino acids include D-stereoisomer forms of natural L-amino acids, as well as synthetic amino acids (those that have non-naturally occurring side chains). In one mode of practice, the cyclic peptide includes a D-amino acid, such as D-phenylalanine, and an adjacent synthetic amino acid. In the step of coupling, a dipeptide including an N-terminal acidic amino acid residue and a C-terminal synthetic amino acid is coupled to a N-terminal D-amino acid of the second peptide fragment to form a third peptide fragment.

The method of the present invention is exemplified in the synthesis of cyclic melanocortin-4 (MC-4) receptor agonist peptides that selectively stimulate MC-4 receptor activity. MC-4 receptor agonist peptides are believed to be useful in treating or preventing obesity (Huzar, D., et al. (1997) Cell 88:131-41) and male erectile dysfunction (MED) (Sebhat, I. K., et al. (2002) J Med. Chem. 45:4589-93). The synthesis of short cyclic MC-4 peptides, which include a non-natural amino acid and have high selectivity for the MC-4 receptor have been described in U.S. Pat. No. 7,045,591. These peptides are represented by the formula: cyclo(Asp-Lys) pentanoyl-Asp-(AAₙₙ)-D-Phe-Arg-Trp-Lys-NH₂ (SEQ ID NO:2), wherein AAₙₙ represent a non-natural amino acid structure as described in the U.S. Pat. No. 7,045,591 patent. Preferred MC-4 agonist peptides have a non-natural amino acid selected from 4-amino-1-phenylpiperidine-4-carboxylic acid and 4-amino-1-(2-methylphenyl)piperidine-4-carboxylic acid.

In one aspect, the invention provides a novel dipeptide. This dipeptide can be used as an intermediate peptide for the synthesis of a cyclic MC-4 peptide, as described herein. The dipeptide is an aspartic acid dipeptide of the Formula I:

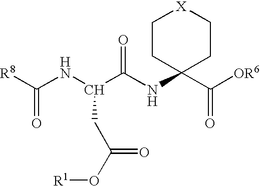

wherein

R¹ is an alkyl protecting group;

X is:

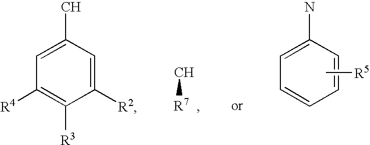

R², R³ and R⁴ are independently hydrogen or a linear or branched alkoxy having from 1 to 4 carbon atoms, wherein when R³ is alkoxy, R² and R⁴ are both hydrogen. R⁵ is hydrogen, linear or branched alkyl having from 1 to 3 carbons, linear or branched alkoxy having from 1 to 3 carbons, or unsubstituted phenoxy. R⁷ is cyclohexyl, cycloheptyl, or a branched alkyl having from 3 to 8 carbon atoms;

R⁸ is alkyl having from 1 to 5 carbon atoms, alkenyl having from 2 to 5 carbon atoms, or alkynyl having from 2 to 5 carbon atoms; and

R⁶ is H or a halogen.

In some aspects the dipeptide is used in a method of forming a cyclic melanocortin-4 receptor agonist peptide. The method comprises steps of synthesizing an aspartic acid dipeptide of Formula I on a resin. Next the aspartic acid dipeptide is cleaved from the resin. A second peptide fragment comprising the sequence: D-Phe-Arg-Trp-Lys, which is attached to a resin is then provided. Next, the carboxyl terminus of the dipeptide is coupled to the amino terminus of the second peptide fragment, thereby forming a peptide having sequence [Formula I]-D-Phe-Arg-Trp-Lys. The peptide is then cyclized by covalently coupling the side chain of the aspartic acid residue with the side chain of the lysine residue. This approach avoids formation of a dead-end urea intermediate otherwise formed by coupling the synthetic amino acid (of the dipeptide) in monomer form.

Compounds produced according to the methods of the invention can be used in pharmaceutical compositions for the treatment of elevated body weight in a subject.

### DETAILED DESCRIPTION OF THE INVENTION

All publications and patents mentioned herein are hereby incorporated by reference in their respective entireties. The publications and patents disclosed herein are provided solely for their disclosure. Nothing herein is to be construed as an admission that the inventors are not entitled to antedate any publication and/or patent, including any publication and/or patent cited herein.

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

The method of the present invention follows a general approach for making cyclic peptides. This includes steps of (a) preparing a first dipeptide comprising an amino acid residue with a first side chain by solid phase synthesis, (b) cleaving the dipeptide from the resin, (c) preparing a second peptide fragment by solid phase synthesis comprising an amino acid residue with a second side chain, (d) coupling the carboxyl terminus of the cleaved dipeptide fragment to the amino terminus of the second peptide fragment while on the resin, thereby forming a third peptide. The third peptide is then (e) cyclized by covalently coupling the first side chain of the dipeptide portion with the second side chain of the second peptide portion. Typically, the third peptide is cleaved from the resin and then cyclized.

The amino acid side chains, including the first and second amino acid side chains (which are covalently coupled during the cyclization step) include protecting groups that are not removed until a point after the dipeptide has been coupled to the second peptide. In many modes of practice, the side chain protecting groups are removed during the step of cleavage of the third peptide from the resin. For example, the resin bound third peptide can be treated with trifluoroacetic acid to remove acid-labile side chain protecting groups and cleave the acid-labile group which links the third peptide to the resin.

According to the invention, a “cyclic peptide” refers to a peptide having at least one pair of amino acid side chains that are covalently coupled. For example, a pair of side chains that are coupled can include a covalent bond formed between a reactive side chain of one amino acid (e.g., of the dipeptide portion of the peptide) and a reactive side chain of another amino acid. A reactive amino acid amino acid side chain can include acidic, basic, or sulfur-containing groups. An exemplary cyclic peptide includes the formation of an amide bond between the side chain of an acidic amino acid (such as aspartic acid or glutamic acid) and the side chain of a basic amino acid (such as lysine, arginine, tryptophan, or histidine).

Cyclic peptides can also be prepared by the formation of a disulfide bond. A disulfide bond is formed through the oxidative coupling of two cysteine residues in the peptide.

The process of the invention is carried out so that the relevant amino acids are in positions in the third peptide so their side chains can be induced to undergo intramolecular amide bond or disulfide bond formation when desired. In some cases the bond is formed between two amino acids that are within about six amino acids of each other. In some cases the bond is formed between the N-terminal and C-terminal amino acids of the peptide.

As a general matter, the method of the present invention can be used in a process to prepare a cyclic peptide of any desired length. For example, the third peptide can be formed by coupling the dipeptide to a second peptide having two or more amino acids, such as a peptide having a number of amino acids in the range of 2 to 10 amino acids. The second peptide can be a tripeptide or tetrapeptide. In one mode of practice, the dipeptide is coupled to a tetrapeptide to form a hexapeptide.

The third peptide can be synthesized as a full-length peptide (referring to peptides wherein no additional amino acids or peptide fragments are coupled to the peptide) or can be synthesized as an intermediate peptide. Intermediate peptides can be subject to one or more coupling steps with additional amino acids or peptide intermediate fragments to produce a peptide of greater length. For example, the third peptide, when formed and cyclized, can be an intermediate compound in that it is coupled to other chemical moieties. These other chemical moieties can be other peptides or types of polymers. For example the third peptide can be coupled to a hydrophilic polymer such as polyethylene glycol (PEG).

The amino acids from which the peptide can be derived can be naturally occurring amino acid residues, non-natural amino acid residues, or combinations thereof. The twenty common naturally-occurring amino acid residues are as follows: A (Ala, alanine), R (Arg, arginine); N (Asn, asparagine); D (Asp, aspartic acid); C (Cys, cysteine) Q (Gln, glutamine), E (Glu, glutamic acid); G (Gly, glycine); H(H is, histidine); I (Ile, isoleucine); L (Leu, leucine); K (Lys, lysine); M (Met, methionine); F (Phe, phenylalanine); P (Pro, proline); S (Ser, serine); T (Thr, threonine); W (Trp, tryptophan); Y (Tyr, tyrosine); and V (Val, valine). Naturally occurring rare amino acids are also contemplated and include, for example, selenocysteine and pyrrolysine.

In some aspects, non-natural amino acids are included in the cyclic peptide. Non-natural amino acids include organic compounds having a similar structure and reactivity to that of naturally-occurring amino acids and include, for example, D-amino acids, beta amino acids, omega-amino acids (such as 3-aminopropionic acid, 2,3-diaminopropionic acid, 4-aminobutyric acid, and the like), gamma amino acids, cyclic amino acid analogs, propargylglycine derivatives, 2-amino-4-cyanobutyric acid derivatives, Weinreb amides of α-amino acids, and amino alcohols. In one aspect of the invention, and as described herein, a non-natural amino acid as described in U.S. Pat. No. 6,600,015 or 7,045,591 is used in the present methods in the synthesis of an arginine-containing peptide.

Residues of one or more other monomeric, oligomeric, and/or polymeric constituents optionally can be incorporated into the cyclic peptide. Non-peptide bonds may also be present. These non-peptide bonds can be between amino acid residues, between an amino acid and a non-amino acid residue, or between two non-amino acid residues. These alternative non-peptide bonds can be formed by utilizing reactions well known to those in the art, and may include, but are not limited to, imino, ester, hydrazide, semicarbazide, azo bonds, and the like.

The invention also contemplates methods of preparing cyclic peptides that have been chemically altered to contain one or more chemical groups other than amino acid residues, sometimes referred to as modified peptides. Such chemical groups can be included at the amino termini of the peptides, the carboxy termini, and/or at one or more amino acid residues along the length of the peptide. In still further embodiments, the peptide can include additional chemical groups present at their amino and/or carboxy termini, such that, for example, the stability, reactivity and/or solubility of the peptides are enhanced. Techniques for introducing such modifications are well known in the art.

The process for solid phase synthesis of the second peptide typically involves the coupling of side chain protected amino acids to a nascent peptide chain, which is resin bound.

According to the invention, the dipeptide and the second peptide are synthesized on a solid phase resin. In some modes of practice, the dipeptide and second peptide are synthesized using standard Fmoc protocols. See, for example, Carpino et al. (1970), J. Am. Chem. Soc. 92(19):5748-5749; Carpino et al. (1972), J. Org. Chem. 37(22):3404-3409, “Fmoc Solid Phase Peptide Synthesis,” Weng C. Chan and Peter D. White Eds. (2000) Oxford University Press Oxford Eng.

Any type of support suitable in the practice of solid phase peptide synthesis can be used. In preferred embodiments, the support comprises a resin that can be made from one or more polymers, copolymers or combinations of polymers such as polyamide, polysulfamide, substituted polyethylenes, polyethyleneglycol, phenolic resins, polysaccharides, or polystyrene. The polymer support can also be any solid that is sufficiently insoluble and inert to solvents used in peptide synthesis. The solid support typically includes a linking moiety to which the growing peptide is coupled during synthesis and which can be cleaved under desired conditions to release the peptide from the support. Suitable solid supports can have linkers that are photo-cleavable, TFA-cleavable, HF-cleavable, fluoride ion-cleavable, reductively-cleavable; Pd(0)-cleavable; nucleophilically-cleavable; or radically-cleavable. Preferred linking moieties are cleavable under conditions such that the cleaved peptide is still substantially globally protected.

In one preferred method of synthesis, the dipeptide is synthesized on an acid sensitive solid support that includes trityl groups, and more preferably on a resin that includes trityl groups having pendent chlorine groups, for example a 2-chlorotrityl chloride (2-CTC) resin (Barlos et al. (1989) Tetrahedron Letters 30(30):3943-3946). Examples also include trityl chloride resin, 4-methyltrityl chloride resin, 4-methoxytrityl chloride resin, 4-aminobutan-1-ol 2-chlorotrityl resin, 4-aminomethylbenzoyl 2-chlorotrityl resin, 3-aminopropan-1-ol 2-chlorotrityl resin, bromoacetic acid 2-chlorotrityl resin, cyanoacetic acid 2-chlorotrityl resin, 4-cyanobenzoic acid 2-chlorotrityl resin, glicinol 2-chlorotrityl resin, propionic 2-chlorotrityl resin, ethyleneglycol 2-chlorotrityl resin, N-Fmoc hydroxylamine 2-chlorotrityl resin, and hydrazine 2-chlorotrityl resin. Some preferred solid supports include polystyrene, which can be copolymerized with divinylbenzene, to form support material to which the reactive groups are anchored. Peptide material typically is attached to the resin beads both at the bead surfaces and within the bead interiors. Fmoc and side chain protected peptide is readily cleaved in a protected state from this resin using mildly acidic reagents such as dilute TFA in DCM or acetic acid.

In some modes of practice the second peptide is synthesized on a resin that allows the formation of a C-terminal amide group following resin cleavage. In one preferred mode of practice, the second peptide is prepared on an Fmoc Rink Amide MBHA resin. This type of resin can be used for the synthesis of peptide amides using Fmoc chemistry, and is designed to allow the attachment of carboxylic acids which are later cleaved as amides.

To further facilitate discussion of the invention, the term “resin,” in the context of the following discussion, generally refers to resin with coupled nascent peptide, unless otherwise noted. Therefore, a step of contacting a resin with a reagent is generally performed to affect the nascent peptide.

For solid phase synthesis, an appropriate reaction vessel can be chosen, depending on the desired quantity of cyclic peptide to be synthesized. Scaled up synthesis of peptide can be carried out in reaction vessels having features including filters, stirrers, temperature gauges, heating and/or cooling elements, reagent input and product export ports and conduits, and inert gas inlet/bubbler mechanisms.

The reaction vessel can be pre-treated prior to addition of the resin in order to prevent reagents from non-specifically adhering to the interior walls of the vessel. For example, silanization reagents, such as dichlorodimethylsilane, can be added to the vessel along with a solvent, such as one that is compatible with the resin and that will be used during solid phase synthesis, such as dichloromethane (DCM). After pre-treatment the vessel can be washed to remove residual silanization reagents.

In order to provide a support having a first coupled amino acid, the resin can be prepared, by example, washing, and then incubating with a solution containing an activated, protected amino acid. The first amino acid and subsequent amino acids that are coupled to the resin typically include an N-terminal protecting group, a side chain protecting group (depending on the specific amino acid), and a group that is reactive with a group pendant from the resin, or a group that is reactive with the pendent amino acid.

In preferred aspects, the first amino acid is attached to the support at the carboxy end, while the N-terminus and side chain groups are protected, as appropriate, by protecting groups. As exemplary description, solid phase synthesis of the FRWK (SEQ ID NO:1) second peptide (a tetrapeptide) is carried from the carboxy-terminal to amino-terminal direction by first loading a protected lysine acid residue onto a Knorr (Fmoc Rink Amide MBHA) resin.

The nature and use of protecting groups is well known in the art. Generally, a suitable protecting group is any sort of group that can help prevent the atom or moiety to which it is attached, e.g., oxygen or nitrogen, from participating in undesired reactions during processing and synthesis. Protecting groups include side chain protecting groups and amino- or N-terminal protecting groups. Protecting groups can also prevent reaction or bonding of carboxylic acids, thiols and the like.

An amino-terminal protecting group includes a chemical moiety coupled to the alpha amino group of an amino acid. Typically, the amino-terminal protecting group is removed in a deprotection reaction prior to the addition of the next amino acid to be added to the growing peptide chain, but can be maintained when the peptide is cleaved from the support. The choice of an amino terminal protecting group can depend on various factors, for example, the type of synthesis performed and the desired intermediate product or final product. As described in the modes of the present invention, Fmoc amino-terminal protecting groups are used for the synthesis of the dipeptide and the second peptide.

A side chain protecting group refers to a chemical moiety coupled to the side chain (i.e., R group in the general amino acid formula H₂N—C(R)(H)—COOH) of an amino acid that helps to prevent a portion of the side chain from reacting with chemicals used in steps of peptide synthesis, processing, etc. The choice of a side chain-protecting group can depend on various factors, for example, the type of synthesis performed, processing to which the peptide will be subjected, and the desired intermediate product or final product. The nature of the side chain protecting group also depends on the nature of the amino acid itself. Generally, a side chain protecting group is chosen that is not removed during deprotection of the α-amino groups during the solid phase synthesis. Therefore the α-amino protecting group and the side chain protecting group are typically not the same.

In some cases, and depending on the type of reagents used in solid phase synthesis and other peptide processing, an amino acid may not require the presence of a side-chain protecting group. This is typically the case when the side chain is non-reactive under standard synthesis conditions. Such amino acids typically do not include a reactive oxygen, nitrogen, or sulfur in the side chain. Amino acids that do not include a reactive oxygen, nitrogen, or sulfur in the side chain are glycine, alanine, leucine, isoleucine, phenylalanine, and valine.

Examples of side chain protecting groups include acetyl(Ac), benzoyl(Bz), tert-butyl, triphenylmethyl(trityl), tetrahydropyranyl, benzyl ether (Bzl) and 2,6-dichlorobenzyl (DCB), t-butoxycarbonyl (Boc), nitro, p-toluenesulfonyl(Tos), adamantyloxycarbonyl, xanthyl(Xan), benzyl, 2,6-dichlorobenzyl, methyl, ethyl and t-butyl ester, benzyloxycarbonyl(Z), 2-chlorobenzyloxycarbonyl(2-Cl-Z), Tos, t-amyloxycarbonyl(Aoc), aromatic or aliphatic urethane-type protecting groups, photolabile groups such as nitro veritryl oxycarbonyl (NVOC); and fluoride labile groups such as trimethylsilylethoxycarbonyl (TEOC).

Preferred side chain protecting groups include t-Bu group for Tyr(Y), Thr(T), Ser(S) and Asp(D) amino acid residues; the trt group for His(H), Gln(O) and Asn(N) amino acid residues; and the Boc group for Lys(K) and Trp(W) amino acid residues. Any one or more of the side-chains of the amino acid residues of the peptide may be protected with standard protecting groups such as t-butyl (t-Bu), trityl (trt) and t-butyloxycarbonyl (Boc).

In other aspects of the invention, the method for synthesizing the second peptide comprises one or more steps of coupling a side chain protected amino acid having an acid-removable alpha amino protecting group. In these aspects, the side chain protecting group is not removable under conditions that are used to remove the acid removable alpha amino protecting group. For example, the side chain protecting group should be compatible with alpha amino protected Boc amino acid chemistry.

According to the invention, the side chain protecting groups are typically retained on the dipeptide and the second peptide throughout solid phase synthesis and also into solid phase coupling of the dipeptide to the second peptide. (Generally, after the solid phase coupling step is completed, a deprotection step is performed to remove one or more protecting groups from the peptide and cleave the peptide from the resin.)

In order to prepare a resin for solid phase synthesis, the resin can be pre-washed in a solvent. For example, a solid phase resin such as a Knorr resin is added to a peptide chamber and pre-washed with a suitable solvent. The washing can be performed to prepare the resin for contact with the first amino acid to be coupled to the resin. In essence, a pre-wash can be performed to promote efficient coupling of the first amino acid to the resin. The pre-wash solvent may be chosen based on the type of solvent (or mixture of solvents) that is used in the coupling reaction, or vice versa.

For resins that include an N-terminal protecting group that is to be removed prior the subsequent steps of coupling amino acids, the washes can be performed in the presence of a compound that cleaves the protecting group from the resin. For example, Fmoc-protected Knorr resin can be deprotected with a piperidine/DMF mixture.

Solvents that are suitable for washing, and also the subsequent coupling reaction include dichloromethane (DCM), dichloroethane (DCE), dimethylfommamide (DMF), and the like, as well as mixtures of these reagents. Other useful solvents include DMSO, pyridine, chloroform, dioxane, tetrahydrofuran, ethyl acetate, N-methylpyrrolidone, and mixtures thereof. In some cases coupling can be performed in a binary solvent system, such as a mixture of DMF and DCM.

In some modes of practice, the second peptide is prepared by loading protected amino acids on the resin or on the nascent peptide chain in an amount of about 1.5 equivalents of amino acid per mole of resin.

The coupling reaction can be performed in the presence of one or more compounds that enhance or improve the coupling reaction. Compounds (coupling reagents) that can increase the rate of reaction and reduce the rate of side reactions include phosphonium and uronium salts that can, in the presence of a tertiary base, for example, diisopropylethylamine (DIEA) and triethylamine (TEA), convert protected amino acids into activated species (for example, BOP, PyBOPO, HBTU, and TBTU all generate HOBt esters). Other coupling reagents typically require help to prevent racemization by adding a protecting reagent. These reagents include carbodiimides (for example, DCC or WSCDI), which typically require an added auxiliary nucleophile (for example, 1-hydroxy-benzotriazole (HOBt), 1-hydroxy-azabenzotriazole (HOAt), or HOSu).

Coupling completion can be monitored with a qualitative ninhydrin test. After the coupling is determined to be complete, the coupling reaction mixture is washed with a solvent, and the coupling cycle is repeated for each of the subsequent amino acid residues of the peptide material. Following the final coupling cycle, the resin is washed with a solvent such as DMF.

In order to couple the next amino acid, removal of the N-terminal protecting group (for example, an Fmoc group) is typically accomplished by treatment with a reagent that includes 20-50% (on a volume basis) piperidine in a solvent, such as dimethylformamide (DMF). After removal of the Fmoc protecting group, several washes are typically performed to remove residual piperidine and Fmoc by-products (such as dibenzofulvene and its piperidine adduct).

After the first amino acid has been coupled to the resin at a desired loading factor and the N-terminal protecting group has been removed, subsequent amino acids can be added to prepare the peptide intermediate fragments. The subsequent amino acids can be utilized at a stoichiometric excess in relation to the loading factor. Preferably the amount of amino acids used in the coupling step is 1.3 equivalent (0.3 excess) or more, and most preferably about 1.5 equivalent (0.5 excess). This excess can also help the reaction tolerate residual base from the deprotection reagent.

The steps of coupling, washing, N-terminal group deprotecting, and washing can be repeated until the desired second peptide is formed, such as the FRWK (SEQ ID NO:1). Following solid phase synthesis the second peptide is maintained on the resin so that it can be coupled in a solid phase reaction to the dipeptide.

The dipeptide of the present invention includes an amino acid residue having a side chain that is subsequently coupled (after the third peptide is formed) to a side chain of an amino acid residue of the second peptide.

In some aspects of the invention, the dipeptide comprises a non-natural amino acid. An exemplary dipeptide includes an aspartic acid residue and a non-natural amino acid as described in U.S. Pat. No. 6,600,015 or 7,045,591. The dipeptide can be coupled to the resin bound FRWK second peptide to provide a MC-4 receptor peptide, which then can be cyclized.

In this aspect the dipeptide can include a compound of the Formula I:

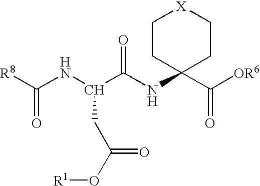

wherein

R¹ is an alkyl protecting group;

X is:

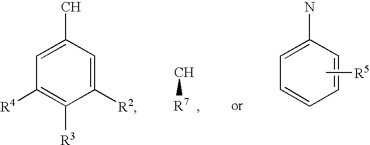

R², R³ and R⁴ are independently hydrogen or a linear or branched alkoxy having from 1 to 4 carbon atoms, wherein when R³ is alkoxy, R² and R⁴ are both hydrogen. R⁵ is hydrogen, linear or branched alkyl having from 1 to 3 carbons, linear or branched alkoxy having from 1 to 3 carbons, or unsubstituted phenoxy. R⁷ is cyclohexyl, cycloheptyl, or a branched alkyl having from 3 to 8 carbon atoms;

R⁸ is alkyl having from 1 to 5 carbon atoms, alkenyl having from 2 to 5 carbon atoms, or alkynyl having from 2 to 5 carbon atoms; and

R⁶ is H or a halogen.

Preferably, in the dipeptide X is

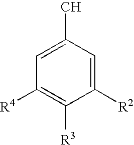

and R³ is alkoxy, and R² and R⁴ are both hydrogen. If R³ is OCH₃, the non-natural amino acid is 1-amino-4-(4-methoxyphenyl)cyclohexane-1-carboxylic acid (4MeOAPC).

In some aspects, R¹ is a branched alkyl group having 4-8 carbon atoms, such as a t-butyl group.

In some aspects, R⁸ is an alkyl group, such as a C4 alkyl group.

In one particular aspect, the dipeptide comprises pentanoyl-Asp-(OtBu)-4MeO-APC—OH.

In one exemplary mode of practice, the dipeptide is by synthesized by solid phase synthesis. The synthesis comprises coupling an amino-protected non-natural amino acid, such as Fmoc-4-MeO-Apc-OH to a resin suitable for Fmoc synthesis, such as 2-CTC resin. Standard coupling and resin washing is performed followed by treatment with piperidine to remove the Fmoc group. Next, an amino- and side chain protected amino acid, such as Fmoc-L-Asp(OtBu)-OH is coupled to the non-natural amino acid. Again, standard coupling and resin washing is performed followed by treatment with piperidine to remove the Fmoc group.

Following Fmoc removal the N-terminus is capped with an alkanoyl group. For example, the N-terminus is treated with a fatty acid anhydride. A suitable fatty acid anhydride is valeric anhydride, which provides the N-terminus with a pentanoyl group.

In order to remove the dipeptide from the resin, a cleaving treatment is carried out in a manner such that the cleaved dipeptide still bears a side chain protecting group. Leaving the protective groups in place helps to prevent undesirable coupling or other undesirable reactions of the dipeptide after cleaving. In the case when Fmoc or a similar chemistry is used to synthesize the peptide, protected cleaving may be accomplished in any desired fashion such as by using a relatively weak acid reagent such as acetic acid or dilute TFA in a solvent such as DCM, which can also swell the resin, being useful for cleavage and separation process. The use of 0.5 to 10 weight percent, preferably 1 to 3 weight percent TFA in DCM is preferred.

After the dipeptide has been cleaved from the resin, a compound can be added to the cleaved dipeptide composition in an amount sufficient to quench the cleavage reaction. For example, in one mode of practice, pyridine (the quenching compound) is added to the composition in an amount approximately double the amount of TFA added to the preceding cleavage reaction. The dipeptide product can then be concentrated in the solvent and extraction with an aqueous liquid used to separate undesired by-products from the dipeptide.

In order to provide the third peptide, the dipeptide is coupled to the second peptide, which is resin bound. In an exemplary process the dipeptide pentanoyl-Asp-(OtBu)-4MeO-APC—OH is coupled to the carboxyl terminus of the second peptide: (D)Phe-Arg-Trp-Lys-resin, wherein the side chains of the amino acids of the second peptide are protected (with the exception of Phe). An exemplary coupling process utilizes HOBT, HBTU, and DIEA in a solvent such as DMF and DCM. Coupling can be performed for a period of time sufficient to generate a negative ninhydrin test (such as overnight).

The resin coupled third peptide (i.e., the dipeptide coupled to the resin-bound second peptide) is then cleaved from the resin using a concentrated TFA solution. Steps of cleaving the third peptide from the solid phase resin can proceed along the lines of the exemplary process as follows. However, any suitable process that effectively cleaves the third peptide from the resin can be used. For example, approximately 5 to 20, preferably about 10 volumes of a solvent containing an acidic cleaving reagent is added to the vessel. The resin beads are immersed in the reagent as a consequence. The cleaving reaction occurs as the liquid contents are agitated at a suitable temperature for a suitable time period. Agitation helps prevent the beads from clumping. Suitable time and temperature conditions will depend upon factors such as the acid reagent being used, the nature of the peptide, the nature of the resin, and the like.

In one mode of practice the third peptide is cleaved from the resin with stirring at from about 15° C. to about 30° C., preferably from about 20° C. to about 25° C. for about 2-3 hours.

Cleavage using a concentrated acidic solution also results in the loss of the amino acid side chain protecting groups.

Following cleavage, the peptide is precipitated. In some modes of practice, a liquid such as methyl-tert-butyl ether (MTBE) is added to the cleaved peptide to cause its precipitation.

Following precipitation the precipitated peptide can be washed with a composition of the precipitating liquid and dilute base. In one mode of practice, the precipitated peptide is washed with a composition of 2% DIEA in MTBE. The base wash facilitates the subsequent cyclization process by minimizing or eliminating formation of a TFA amide on the lysine side chain.

Precipitated peptide solids can then be dried.

Precipitated peptide can then be dissolved in a suitable solvent and subjected to a cyclization reaction. If the cyclization process is directed to the formation of an amide bond between the side chain of an acidic amino acid (such as aspartic acid or glutamic acid) and the side chain of a basic amino acid (such as lysine, glutamine, or histamine), it can be carried out using reagent common to the coupling process, such as HBTU and DIEA. For the peptide pentanoyl-Asp-(4-MeO-Apc)-D-Phe-Arg-Lys-NH₂ (SEQ ID NO.: 2) cyclization results in the covalent coupling of the aspartic acid and lysine side chains providing cyclo (Asp-Lys) Pentanoyl-Asp-(4-MeO-Apc)-D-Phe-Arg-Lys-NH₂.

In one preferred mode of practice, cyclization is performed using a concentrated peptide solution. For example, the cyclization reaction is performed at a concentration in the range of about 15 g/L to about 25 g/L (peptide/solvent). In one mode of practice, the cyclization reaction can be carried out at a temperature of about 20° C. to about 25° C. for about one hour. Following cyclization, the reaction can be quenched with water.

Following cyclization, the peptide can be subjected to chromatographic purification. The peptide can also be subjected to one or more salt exchanges. For example, the peptide TFA salt can be subjected to salt exchanges to provide peptide-acetate salts, and peptide-lactate salts. This can be accomplished by loading the peptide back on a column and then flushing the column with a desired acetate salt (e.g., ammonium acetate) to elute the peptide. A lactate salt can be formed by mixing a lactic acid solution with the peptide-acetate and then lyophilizing the mixture.

The compounds prepared in according to the methods of the invention can be used to provide selective MC-4 receptor agonist activity in vivo. It is known that agonists of MC4-R activity cause reduction of food intake in a mouse model of human obesity.

Therefore administration of these compounds agonizes MC4-R activity which is important in the regulation of body weight. The pharmaceutical compositions containing the compounds of this invention may be formulated at a strength effective for administration by various means to a human or animal patient experiencing undesirably elevated body weight, either alone or as part of an adverse medical condition or disease, such as type II diabetes mellitus. A variety of administrative techniques can be used. Average quantities of the active compound may vary and in particular should be based upon the recommendations and prescription of a qualified physician or veterinarian.

### EXAMPLE I

### Preparation of Fmoc-D-Phe-Arg(Pbf)-Trp(Boc)-Lys(Boc)-Resin

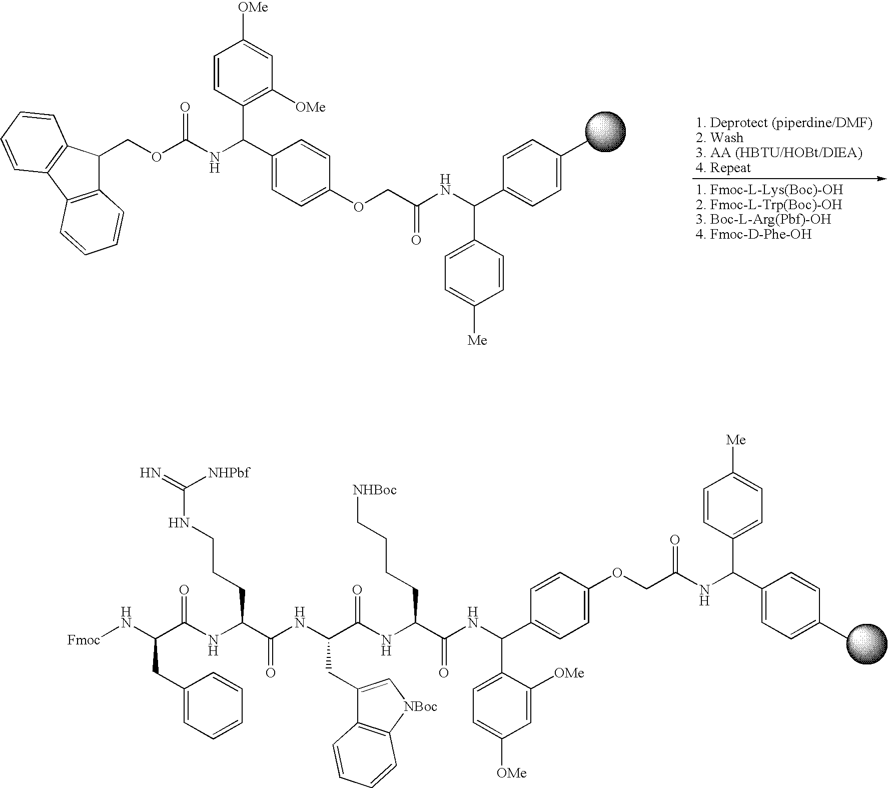

Deprotection Knorr Resin

| | | Charged 6-L SPPS |
|---|---|---|
| 305.38 | g | Knorr resin and |
| 3.6 | L | DMF. Stirred at 100 RPM for 30 min then drained DMF. |
| | | Refilled with |
| 3.0 | L | DMF. The temperature was adjusted to 25° C. |
| | | Drained reactor and deprotected with |
| 2 × 3.6 | L | 20% Piperidine/DMF for 60 min each. Washed resin with |
| 4 × 3.6 | L | DMF |

Couple Fmoc-Lys(Boc)-OH

| | | |
|---|---|---|
| 192.8 | g | Fmoc-L-Lysine(Boc)-OH |
| 63.44 | g | HOBT hydrate |
| 68.0 | g | DIEA |
| 1.7 | L | DMF. The solution was cooled to 5° C. and combined with |
| 157.0 | g | HBTU in |
| 1 | L | DMF and cooled to 5° C. for 15 min. |
| | | The activated ester solution was added to the |
| | | SPPS and rinsed in with |
| 0.6 | L | DCM. The coupling was maintained for 3 h. |
| | | (Reactor vol = 4.7 L). |
| | | Sampled for completion (Kaiser) at 2 and 3 h. Both |
| | | samples were ninhydrin colorless. |
| | | Drained reactor and washed with |
| 4 × 3.6 | L | DMF. Drained reactor and deprotected with |
| 2 × 3.6 | L | 20% Piperidine/DMF for 30 min each. Washed resin with |
| 4 × 3.6 | L | DMF. |

Couple Fmoc-Trp(Boc)-OH

| | | |
|---|---|---|
| 125.32 | g | Fmoc-L-Trp(Boc)-OH |
| 63.44 | g | HOBT hydrate |
| 68.0 | g | DIEA |
| 1.7 | L | DMF. The solution was cooled to 5° C. and combined with |
| 157.0 | g | HBTU in |
| 1 | L | DMF and cooled to 5° C. for 15 min. |
| | | The activated ester solution was added to the SPPS rinsed in with |
| 0.6 | L | DCM. The coupling was maintained for 9 h. (Reactor vol = 5 L). |
| | | Sampled for completion (Kaiser) 3 h. Sample was ninhydrin colorless. |
| | | Drained reactor and washed with |
| 4 × 3.6 | L | DMF. Drained reactor and deprotected with |
| 2 × 3.6 | L | 20% Piperidine/DMF for 30 min each. Washed resin with |
| 4 × 3.6 | L | DMF. |

Couple Fmoc-L-Arg(Pbf)-OH

| | | |
|---|---|---|
| 267.5 | g | Fmoc-L-Arg(Pbf)-OH |
| 63.44 | g | HOBT hydrate |
| 68.0 | g | DIEA |
| 1.7 | L | DMF. The solution was cooled to 5° C. and combined with |
| 157.0 | g | HBTU in |
| 1 | L | DMF and cooled to 5° C. for 15 min. |
| | | The activated ester solution was added to the SPPS and rinsed in with |
| 0.6 | L | DCM. The coupling was maintained for 3 h. (Reactor vol = 5 L). |
| | | Sampled for completion (Kaiser) 3 h. Sample was ninhydrin colorless. |
| | | Drained reactor and washed with |
| 4 × 3.6 | L | DMF. Drained reactor and deprotected with |
| 2 × 3.6 | L | 20% Piperidine/DMF for 30 min each. Washed resin with |
| 4 × 3.6 | L | DMF. |

Couple Fmoc-D-Phe-OH

| | | |
|---|---|---|
| 159.7 | g | Fmoc-D-Phe-OH |
| 63.44 | g | HOBT hydrate |
| 68.0 | g | DIEA |
| 1.7 | L | DMF. The solution was cooled to 5° C. and combined with |
| 157.0 | g | HBTU in |
| 1 | L | DMF and cooled to 5° C. for 15 min. |
| | | The activated ester solution was added to the SPPS and rinsed in with |
| 0.6 | L | DCM. The coupling was maintained for 3 h. (Reactor vol = 5 L). |
| | | Sampled for completion (Kaiser) 3 h. Sample was ninhydrin colorless. |
| | | Drained reactor and washed with |
| 4 × 3.6 | L | DMF. Drained reactor and washed resin with |
| 4 × 2 | L | Methanol. The loaded resin was dried under a nitrogen sweep. A 0.37 g |
| | | sample was taken. |
| | | Weight = 493.81 g (Lot 503-024) |

### EXAMPLE 2

### Preparation of Pentanoyl-Asp-(OtBu)-4-MeO-Apc-OH

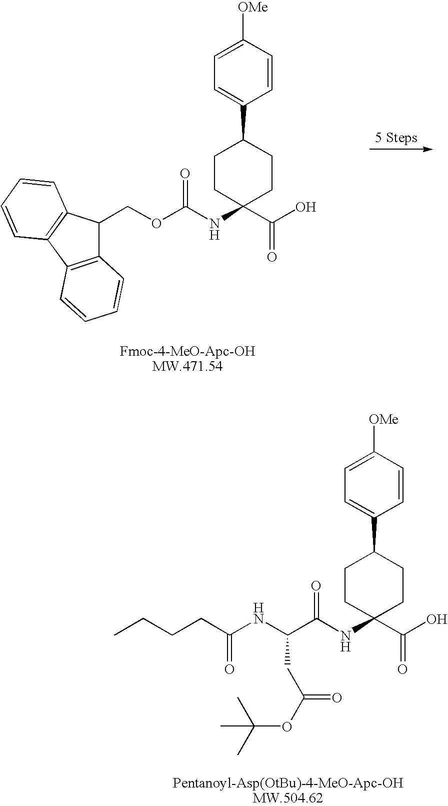

Loading Fmoc-4-MeO-Apc-OH

| | | Charged 6-L SPPS |
|---|---|---|
| 300.07 | g | 2-CTC-Resin and |
| 3 | L | DCM. Stirred 30 min. Made up a solution of |
| 84.87 | g | Fmoc-4-MeO-Apc-OH in |
| 2.1 | L | DMF and |
| 0.3 | L | DCM. The solution was stirred for 30 min and |
| 69.83 | g | DIEA added. The solution was then charged to the swollen resin. Stirring |
| | | was continued for 20 h. The resin was drained and stirred with |
| 3 | L | DMF for 5 min. End capping was achieved by addition of a solution of |
| 0.3 | L | DIEA in |
| 0.27 | L | Methanol and stirring for 1 h. The resin was drained and washed with |
| 3 | L | DMF followed by an additional |
| 1.5 | L | DMF. The resin was then washed with |
| 5 × 3 | L | DCM (The 5^{th} wash was UV negative). The resin was then washed with |
| 3 × 3 | L | DMF. The resin was drained and deprotected with |
| 2 × 2.3 | L | 20% Piperidine/DMF for 30 min each. Washed resin with |
| 5 × 2.3 | L | DMF. A sample was taken for cleavage and determination of loading |
| | | Loading = 0.45 mmol/g |
| | | weight of 352.5 g was obtained. |

Couple Fmoc-L-Asp(OtBu)-OH

| | | |
|---|---|---|
| 129.2 | g | Fmoc-L-Asp(OtBu)-OH |
| 48.2 | g | HOBT hydrate |
| 50.8 | g | DIEA |
| 1.33 | L | DMF. The solution was cooled to 5° C. and combined with |
| 119.2 | g | HBTU in |
| 0.67 | L | DMF and cooled to 5° C. for 15 min. |
| | | The activated ester solution was added to the SPPS |
| | | rinsed in with |
| 0.6 | L | DCM. The coupling was maintained overnight. |
| | | The sample was ninhydrin colorless. Drained reactor and |
| | | washed with |
| 4 × 2.3 | L | DMF. Drained reactor and deprotected with |
| 2 × 2.3 | L | 20% Piperidine/DMF for 30 min each. Washed resin with |
| 5 × 2.3 | L | DMF. |

Capping with Valeric Anhydride

| | | A solution of |
|---|---|---|
| 146.3 | g | Valeric anhydride, |
| 0.274 | L | DIEA in |
| 1.5 | L | DMF was added to the SPPS. The solution was rinsed |
| | | in with |
| 1.2 | L | DMF and the reaction was stirred for 30 min and sampled |
| | | for completion by HPLC. The reaction was complete. |
| | | Drained reactor and washed with |
| 4 × 2.3 | L | DMF. Washed resin with |
| 3 × 2.3 | L | DCM |

Cleavage

| | | Charged SPPS with |
|---|---|---|
| 3 | L | DCM and cooled to 0° C. Drained reactor after cooling complete. Added a |
| | | solution of |
| 30 | mL | Trifluoracetic acid in |
| 3 | L | DCM (1%) and stirred at 0-5° C. for 30 min. Charged |
| 60 | mL | Pyridine and stirred for 5 min. Drained reactor and washed with |
| 4 × 2.3 | L | DCM at 20-25° C. The cleavage solutions were stored at 0-5° C. overnight. |
| | | The DCM was concentrated by distillation in a rotovap with a bath temp |
| | | of 30° C. and 250 Torr vacuum to a volume of ~1 L while feeding |
| 150 | mL | DI water. The DCM/water was transferred to a separatory funnel and the |
| | | aqueous layer removed. The organic layer was washed with |
| 3 × 100 | mL | DI water. The organic layer was combined with |
| 100 | mL | DI water and the DCM removed by distillation in a rotovap with a bath |
| | | temp of 30° C. The vacuum was increased to 100 Torr until no more DCM |
| | | was removed. The contents of the flask solidified. Charged flask with |
| 400 | mL | DCM to dissolve the solids. Transferred to a separatory funnel and |
| | | removed the aqueous layer. Distilled of the DCM while feed stripping |
| 325 | mL | DI water with a bath temp of 30° C. until no more DCM was removed at |
| | | 100 Torr. The solids were collected by filtration and washed with |
| 100 | mL | DI water followed by an additional |
| 50 | mL | DI water. The product was dried under vacuum at 20-25° C. |

| | |
|---|---|
| Weight | = 50.5 g (Ref 503-152) |
| Yield | = 55.6% (from Fmoc-4-MeO-Apc-OH) |
| | = 63.5% (based on loading) |
| Purity | = 96.15% (AN HPLC) |

### EXAMPLE 3

### Preparation of Pentanoyl-Asp(OtBu)-4-MeO-Apc-D-Phe-Arg(Pbf)-Trp(Boc)-Lys(Boc)-Resin

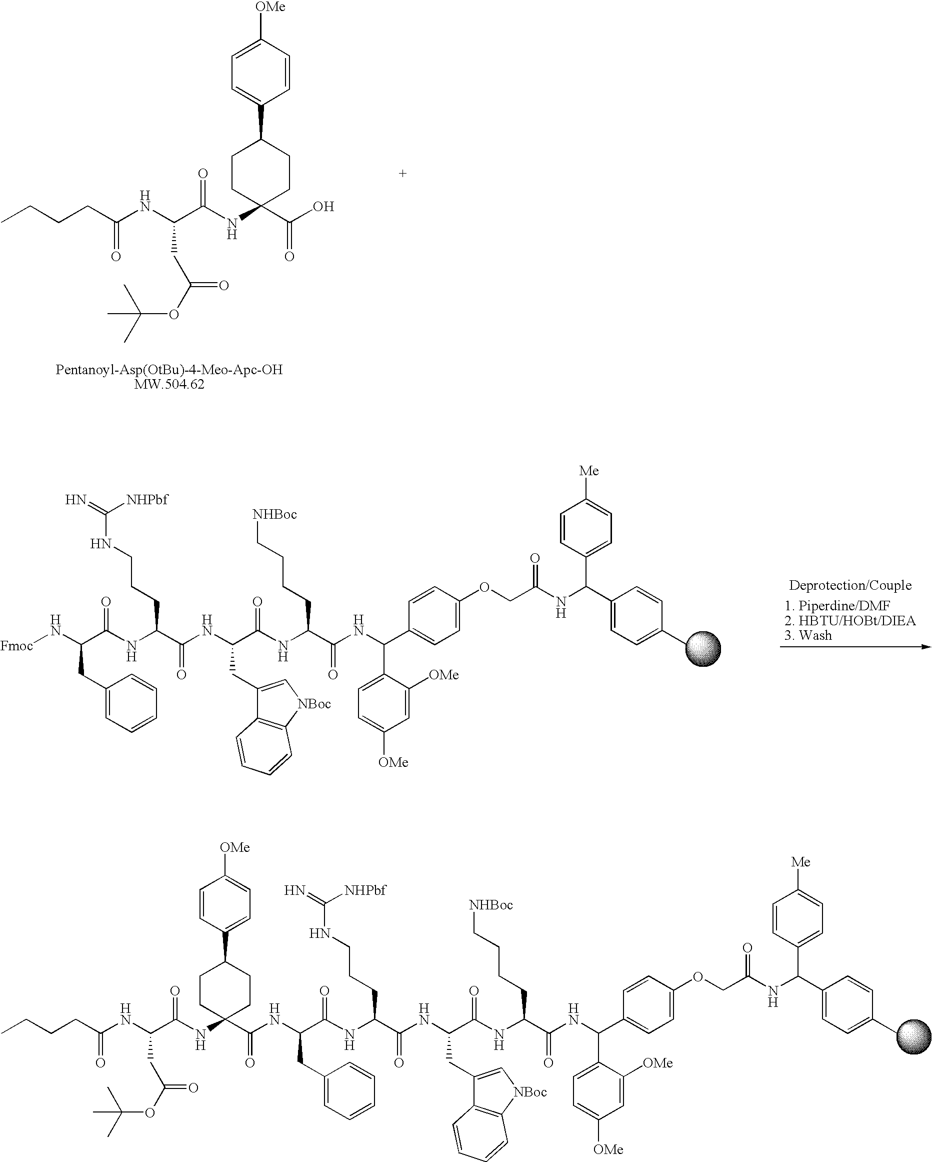

Deprotection of Fmoc-D-Phe-Arg(Pbf)-Trp(Boc)-Lys(Boc)-Resin

| | | Charged 6-L SPPS with |
|---|---|---|
| 452.8 | g | Fmoc-D-Phe-Arg(Pbf)-Trp(Boc)-Lys(Boc)-Resin. Swelled |
| | | the resin with one wash of |
| 3 | L | DCM. Drained the reactor and washed with |
| 4 × 3 | L | DMF. The resin was drained and deprotected with |
| 2 × 3 | L | 20% Piperidine/DMF for 30 min each. Washed resin with |
| 3 × 3 | L | DMF and drain |

Coupling with Pentanoyl-Asp(OtBu)-4-MeO-Apc-OH

| | | |
|---|---|---|
| 159.0 | g | Pentanoyl-Asp(OtBu)-4-MeO-Apc-OH |
| 48.14 | g | HOBT hydrate |
| 45.66 | g | DIEA |
| 1.5 | L | DMF. The solution was cooled to 5° C. and combined with |
| 119.52 | g | HBTU in |
| 1 | L | DMF and cooled to 5° C. for 15 min. |
| | | The activated ester solution was added to the SPPS rinsed in with |
| 0.6 | L | DCM. The coupling was maintained overnight. (Reactor vol ~ 5 L). |
| | | Sampled for completion (Kaiser). Sample was ninhydrin colorless. |
| | | Drained reactor and washed with |
| 3 × 3 | L | DMF. Drained reactor and washed with |
| 73 × 3 | L | DCM. The resin was transferred to a 2-L sintered glass filter and blown |
| | | down with nitrogen. (Note: due to the stainless steel mesh in the 6-L |
| | | SPPS, the resin was transferred to a 2-L SPPS for cleavage) |
| | | Weight = 499.81 g |

### EXAMPLE 4

### Pentanoyl-Asp(OtBu)-4-MeO-Apc-D-Phe-Arg(Pbf)-Trp(Boc)-Lys(Boc)

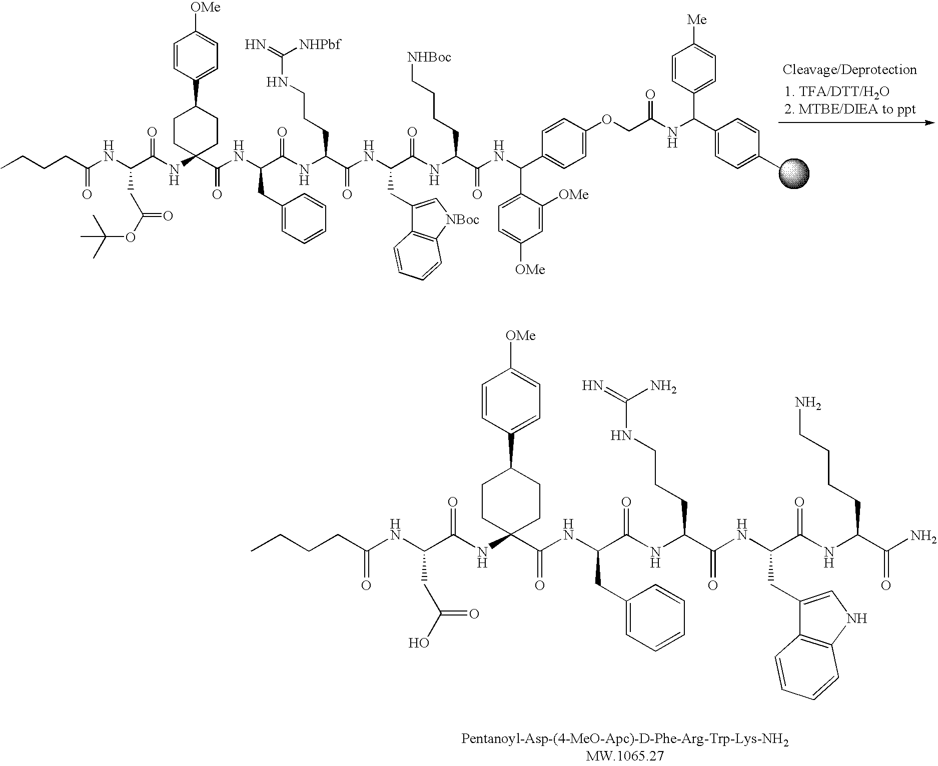

| | | Charged 2-L SPPS with |
|---|---|---|
| 150.08 | g | Pentanoyl-Asp(OtBu)-4-MeO-Apc-D-Phe-Arg(Pbf)-Trp(Boc)-Lys(Boc)- |
| | | Resin and |
| 1 | L | DCM to swell the resin. Prepared cleavage solution from |
| 75.02 | g | DTT |
| 75 | mL | DI water |
| 1.46 | L | TFA. Drained the resin and charged the cleavage solution to reactor. |
| | | Stirred the reactor for 2 h 20 min at 20-25° C. Charged a 5 gallon carboy |
| | | with |
| 12 | L | MTBE and cooled to 0-5° C. Drained the cleavage solution into the 5 |
| | | gallon carboy forming a ppt. Charged the reactor with |
| 500 | mL | TFA and stirred for 5 min followed by the addition of |
| 500 | mL | MTBE. Stirred for 2 min and drained the solution into the carboy. |
| | | The contents of the carboy were well mixed then transferred to 250 mL |
| | | FLPE bottles (8 bottles with ~ 230 mL/bottle). The bottles were |
| | | centrifuged at 2600 RPM for 1 min. The supernatant was decanted and the |
| | | bottles refilled. This process was continued until all the suspension was |
| | | processed. Each bottle was then filled with MTBE (~ 1.8 L total), capped |
| | | and shaken to resuspend the solids and then centrifuged. A solution of |
| 35 | mL | DIEA in |
| 1.46 | L | MTBE was prepared. Approximately |
| 175 | mL | DIEA/MTBE solution was added to each of the 8 bottles. The bottles were |
| | | capped and shaken and stored in the refrigerator at 5° C. overnight. |
| | | The bottles were removed from the refrigerator and centrifuged at 2500 RPM |
| | | and the supernatant decanted. |
| 220 | mL | 2% DIEA in MTBE was added to each bottle. The bottles were capped, |
| | | shaken and centrifuged. The supernatant was decanted and |
| 220 | mL | MTBE was added to each bottle, shaken and decanted. This operation was |
| | | repeated a third time with |
| 220 | mL | MTBE. The resulting wet solids were dried overnight under a vacuum of |
| | | 200 Torr. Note: the vacuum should be applied gradually or bumping with |
| | | loss of peptide will occur. |

Weight of crude dried linear hexapeptide = 47.05 g (Ref 503-102)

### EXAMPLE 5

### Cyclo (Asp-Lys) Pentanoyl-Asp-(4-MeOApc)-D-Phe-Arg-Trp-Lys-NH₂

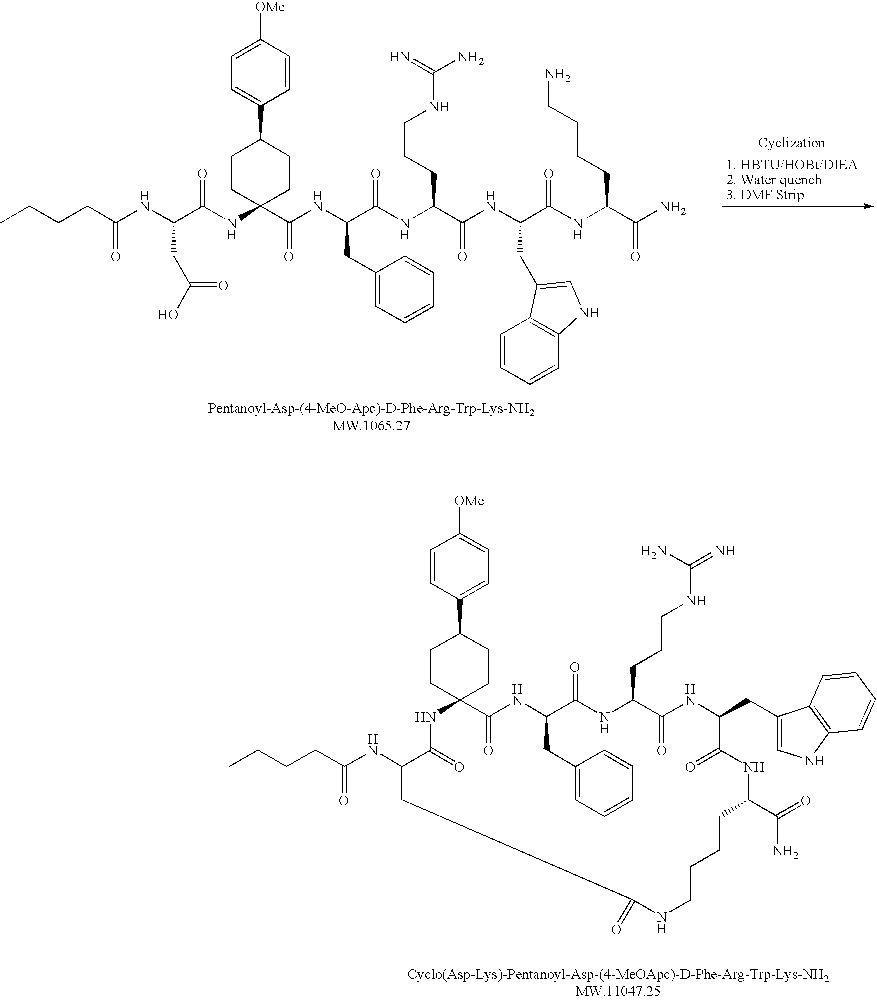 [0097] Equipment: 2-L jacketed vessel, turbine agitator, Nitrogen and vacuum inlets, thermocouple, metering pump

### Procedure

| | | Charged the 2-L vessel with |
|---|---|---|
| 49.46 | g | HBTU and |
| 1.5 | L | DMF. Stirred at 20-25° C. until dissolved. Prepared a solution of |
| 50.0 | g | Pentanoyl-Asp-(4-MeOApc)-D-Phe-Arg-Trp-Lys-NH₂ in |
| 1.5 | L | DMF containing |
| 50 | mL | DIEA. Adjusted the agitation to 139 RPM and metered in the linear |
| | | hexapeptide solution at a rate of 25 mL/min at 20-25° C. ~ 1 h. The |
| | | reaction was sampled for analysis. The reaction was found to be complete |
| | | and |
| 100 | mL | DI water was then added to quench the reaction. The solution was |
| | | transferred in two portions to a 2-L round bottom flask and the solvents |
| | | distilled on a rotovap with a bath temperature of 30° C. under vacuum at a |
| | | pressure of <9 Torr |

Weight of crude product = 154.11 g (Ref 450-269)
Assay = 10% w/w (15.4 g contained peptide)

### EXAMPLE 6

### Chromatographic Purification

### Cyclo (Asp-Lys) Pentanoyl-Asp-(4-MeO-Apc)-D-Phe-Arg-Lys-NH₂ 1:1 Trifluoroacetate

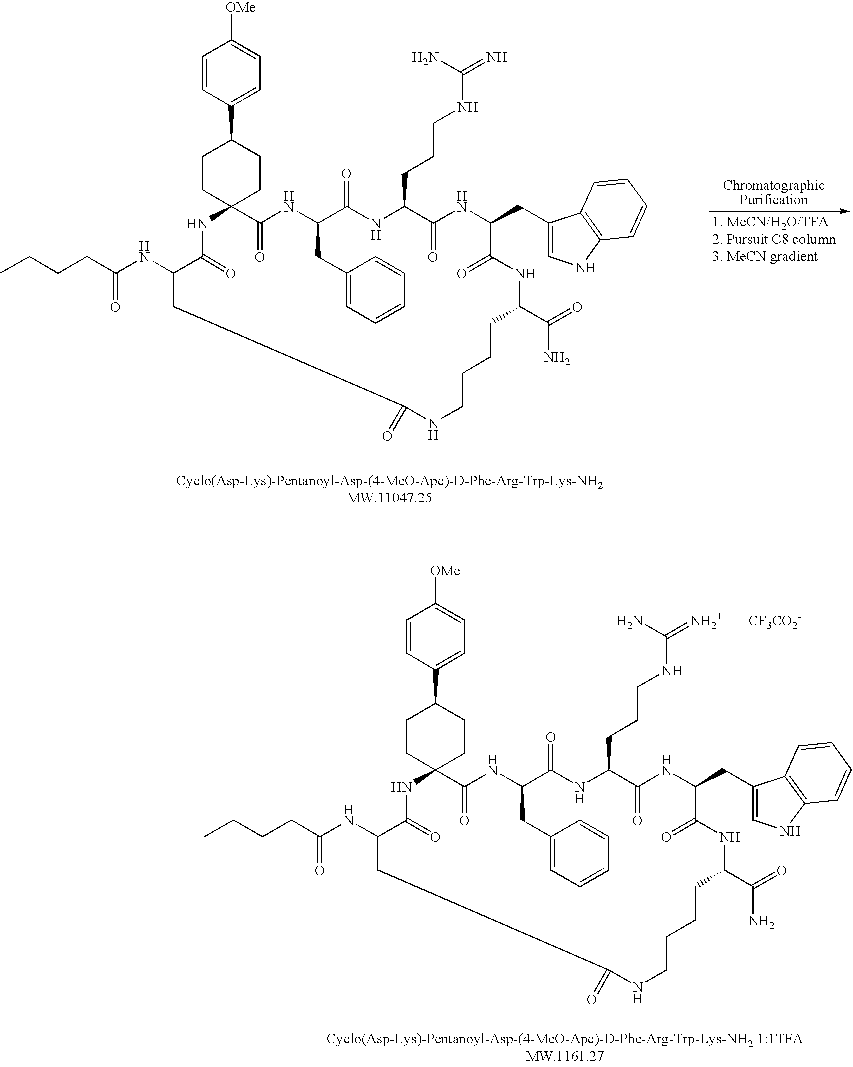

Purification chromatography was performed on three crude batches of cyclic MC-4 hexapeptide for toxicological studies. Purification was performed on a Pursuit C18 10 micron, 50×250 mm column at low pH. The overall contained yield was 85% with an overall purity of 94%. Starting crude purities for three batches were 10 wt % or less. The low crude purity decreased load capacity and gave significant injection solution filtration issues. A total of 35 injections were completed. Of the 35 injections, 28 were crude injections and 7 were recycle injections. The chromatography gave 33.7 g of purified cyclic MC-4 out of the 39.7 g of crude cyclic MC-4 contained. The pooling for this purification was targeted for the mid ninety's for toxicological material. Increasing the purity to the upper nineties will decrease the estimated yield to 50-60% based on fraction analysis in this purification.

### Preparative Chromatography:

Injection solution: Preparation of the cyclic MC-4 crudes into a solids free 1.5 mg/mL (contained cyclic MC-4) injection solution was complicated by the low assay and high salt content of the isolated crudes. (Table 1) Several different filtering configurations were tried with a three tiered stack of glass fiber filters of decreasing pore size giving the best filtration. This filter arrangement using 125 mm diameter filters was successful in filtering 500 mL of injection solution. This required splitting each 1000 mL injection into two separate filtrations and recombining the filtrates for injection.
Buffer Preparation: The preparative buffers were prepared in 20 L carboys for both mobile phase A and mobile phase B. Both mobile phases were 0.1% TFA solutions giving an approximate pH of 2. Two mobile phase solutions were used in cyclic MC-4 purification. All percentages are by volume

### Mobile Phase A is 0.1% TFA in 90/10H₂O/ACN

### Mobile Phase B is 0.1% TFA in 10/90H₂O/ACN

Chromatography Conditions: cyclic MC-4 was purified on a pre-packed Varian Pursuit C18 column, 10 micron, 50×250 mm. Using an Agilent 1100 preparative HPLC equipped with a wide bore solvent switching valve for loading injection solution and a 13 position valve for fraction collection. Each injection varied from 1.0 g to 1.5 g of contained cyclic MC-4 based on the crude assay. Five or more fractions were collected from each injection and were combined based on HPLC fraction analysis. The cycle time per injection was about two hours. The following elution conditions were found to be optimal for the best purification results:

| | |
|---|---|
| Column: | Pursuit C18, 10 micron, 50 × 250 mm |
| Detector: | 280 nm (8 nm bandwidth, 350/20 nm ref) |
| Column Temp: | Ambient |
| Flow rate: | 80 ml/min |
| Mobile phase: | A = 0.1% TFA in 90/10 H₂O/ACN |
| | B = 0.1% TFA in 10/90 H₂O/ACN |
| Sample loading: | Manually via pump A switching valve at 60 mL/min |
| Gradient: | Initial condition 25% B |
| | 0-45 min linear gradient to 57% B |
| | 45.0-45.1 min step to 70% B |
| | 45.1-55.0 min hold 70% B (column flush) |
| | 55.0-52.0 linear gradient to 25% B |
| | 52.0-72.0 25% B (column re-equilibration) |

Recycle injections: Recycle injections for front cuts and back cuts were re-injected by diluting the pooled fractions with equal volumes of water and injecting back onto the column. The same gradient conditions were used for the recycles.
Crude injection solution preparation: Isolated crudes from three lab runs were used in the preparation of the injection solutions. The crude purities ranged from 8% to 10% contained purity. All of the crude batches were highly colored and contained insoluble solids. The desired final filtered concentration of cyclic MC-4 in the injection solution is 1.5 mg/mL (contained). Depending on the w/w assay of the crude, the final actual concentration will be about 15 mg/mL of isolated crude.

TABLE 1

| Assay (% w/w) | Purity (% AN) |
|---|---|
| 8.0 | 30.70 |
| 10.0 | 46.30 |
| 10.5 | 45.30 |

The following procedure was used to prepare 1072 mL of injection solution for filtration: Based on the w/w assay, 1.5 g of contained cyclic MC-4 is dissolved in 160 mL of DMF. While gently stirring the dissolved solution add 160 mL of ACN containing 01% TFA. With continued stirring add 752 mL of water containing 0.1% TFA. This crude solution is then filtered through a triple filter stack of glass fiber filters of decreasing pore size (Whatman).
Top GF/D 2.7 μm pore (1823125)
Middle GF/C 1.2 μm pore (1822125)
Bottom GF/F 0.7 μm pore (1825125)
   This filter arrangement using 125 mm diameter filters is used in vacuum filtering 500 mL of injection solution. This requires splitting each 1000 mL injection into two separate filtrations and recombining the filtrates for injection. The final filtrate was completed at room temperature and immediately before injection. Cooling of the filtrate or extended time standing at ambient temperature, will cause the solution will become cloudy. This procedure may be scaled up or down as needed.

Fractions were taken from the column eluent by a fraction collector using Table 2 time table. These times were adjusted as needed.

TABLE 2

| Time (min) | Description |
|---|---|
| 0.0-23.0 | Waste |
| 23.0-24.5 | Front recycle |
| 24.5-27.0 | Fraction 1 |
| 27.0-30 | Fraction 2 |
| 30.0-33.0 | Fraction 3 |
| 33.0-35.8 | Fraction 4 |
| 35.8-39.0 | Back recycle |
| 39.0+ | Waste |

### EXAMPLE 7

### Salt Exchanges

### Cyclo (Asp-Lys) Pentanoyl-Asp-(4-MeO-Apc)-D-Phe-Arg-Lys-NH₂ 1:1 Acetate

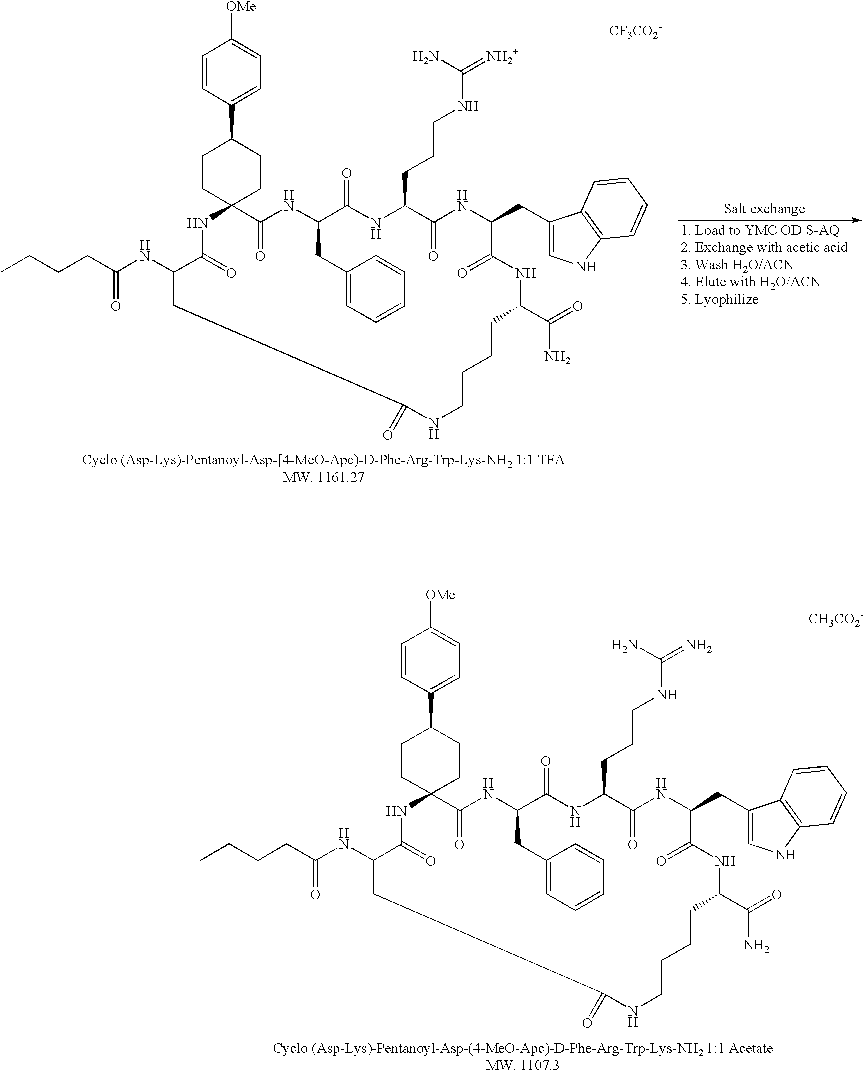

The cyclic MC-4 TFA salt pool was converted to the lyophilized acetate salt

### Equipment

5.0×25 cm Vydak C4,1-micron, 300 Å pre-packed column
Dual pump prep system with adjustable wavelength detector (equivalent to a Varian Prostar system with a Model 210 loading pump, Model 215 elution pumps and a Model 320 detector).

### Mobile Phases

### A: 10% Acetonitrile/Deionized H₂O, 20 mM NH₄OAc

### B: 70% Acetonitrile/Deionized H₂O, 20 mM NH₄OAc

### Pool Preparation

The combined purification pool fractions are diluted 1:1 (to ˜25% Acetonitrile) with Deionized H₂O.

### Pool Loading

Load is approximately 10 g of cyclic MC-4 (˜8 L at ˜1.25 g/L concentration) onto column at 25 mL/min. This takes ˜5 h.

### Elution

After flushing the column with ˜5 equiv of NH₄OAc the product is eluted.
Flow rate 50 mL/min, detector at 280 nm

TABLE 3

| Time (min) | % A | % B |
|---|---|---|
| 0 | 100 | 0 |
| 49 | 100 | 0 |
| 50 | 0 | 100 |
| 80 | 0 | 100 |
| 81 | 100 | 0 |
| 100 | 100 | 0 |

The product peak was collected over 10 minutes for a total volume of 500 mL and lyophilized after partitioning into four tared 500 mL wide mouth poly bottles. The solid product from the 4 bottles was collected.

### Weight=10.49 g

### EXAMPLE 8

### Salt Exchanges

### Cyclo (Asp-Lys) Pentanoyl-Asp-(4-MeO-Apc)-D-Phe-Arg-Lys-NH₂ 1:1 Lactate

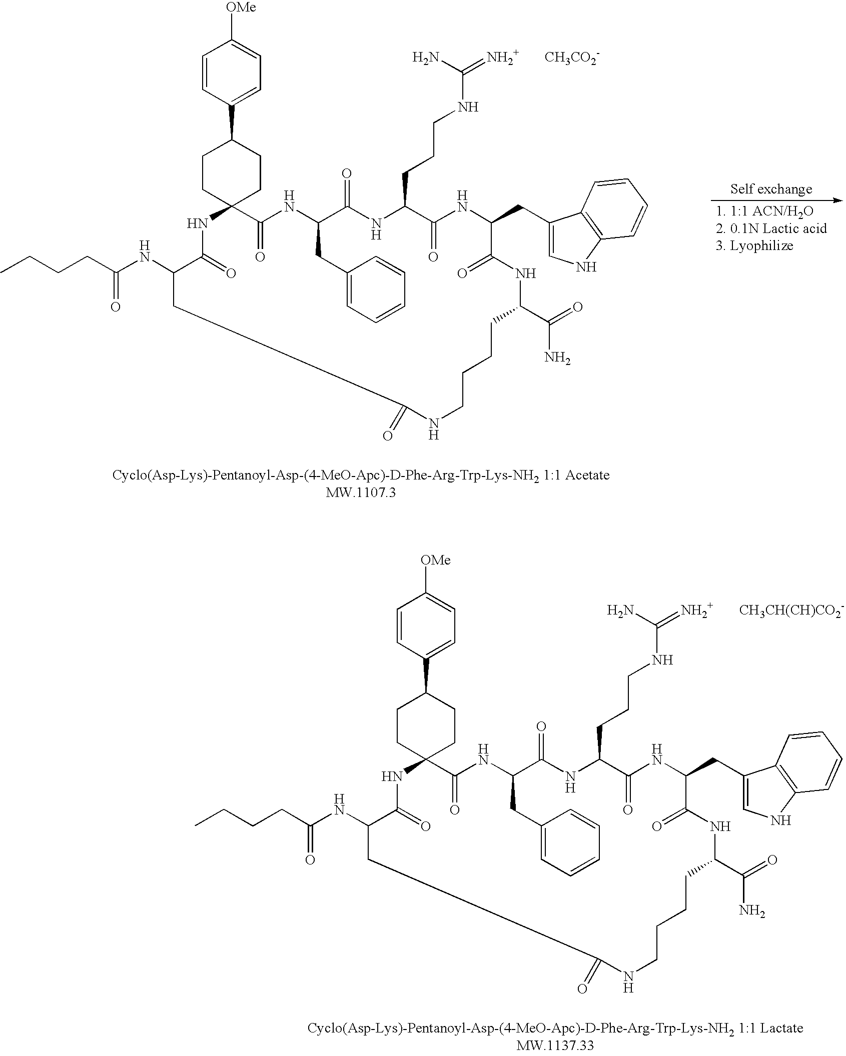

Lactic Acid, Racemic, U.S.P. Spectrum chemical Mfg. Corp. catalog number LI010, CAS 50-21-5, assay 88.0-92.0%
Make up 0.1 N Lactic acid solution
5.0 g of Cyclo (Asp-Lys) Pentanoyl-Asp-(4-MeO-Apc)-D-Phe-Arg-Lys-NH₂ 1:1 Acetate is dissolved in a minimum volume of 1/1 acetonitrile/H₂O; (ACN/H₂O) 40 to 50 mL to which is added 45.154 mL 0.1N lactic acid solution. This solution is further diluted until slightly turbid and then frozen and lyophilized. (Once a solid powder if left under vacuum the amount of acetic acid present will be reduced). The lyophilized powder is analyzed by NMR 2.0 mg in DMSO-D6 with comparison of peaks at δ 1.16 (methyl of lactic acid) and δ 0.82 (terminal CH₃ of pentanoyl) which should be in a ratio of 1:1 (can be measured as mm and ratio obtained). If the ratio is off it can be adjusted by either addition of acetate salt (if too high) of additional 0.1 N lactic acid solution. (5.0 g is the largest scale performed due to equipment size. Two 5.0 g batches were also done with 375 μL of 88.0-92.0% lactate and after 11″ NMR indicated 67-69% lactate. After addition of 200 μL of 88.0-92.0% lactate and after relypholization a final ratio of 1.0 to 1.09 lactate was obtained.)

What is claimed is:

1. A method of forming a cyclic peptide comprising steps of:
preparing a dipeptide fragment on a resin, the dipeptide fragment comprising an acidic amino acid residue comprising a first side chain;
cleaving the first peptide fragment from the resin;
preparing a second peptide fragment on a resin, the second peptide comprising an amino acid residue with a second side chain;
coupling the carboxyl terminus of the dipeptide fragment to the amino terminus of a second peptide fragment, thereby forming a third peptide; and
cyclizing the third peptide by covalently coupling the first side chain of the dipeptide portion with the second side chain of the second peptide portion.

2. The method of claim 1 wherein the dipeptide fragment comprises a carboxyl-terminal non-natural amino acid.

3. The method of claim 1 wherein the dipeptide fragment comprises an amino-terminal aspartic acid residue.

4. The method of claim 3 wherein the dipeptide fragment comprises an aspartic acid dipeptide comprising pentanoyl-Asp-(OtBu)-4-MeO-Apc-OH.

5. The method of claim 1 wherein the second peptide comprises a tetrapeptide.

6. The method of claim 1 wherein the second peptide comprises an amino-terminal D-amino acid residue.

7. The method of claim 6 wherein the second peptide comprises an amino-terminal D-phenylalanine residue.

8. The method of claim 1 wherein the basic amino acid residue of the second peptide comprises a lysine residue.

9. The method of claim 1 comprising a step of cleaving the third peptide from the resin, which is performed prior to the step of cyclizing.

10. An aspartic acid dipeptide of Formula I: 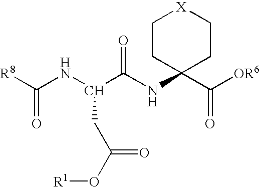
wherein
R¹ is an alkyl protecting group;
X is: 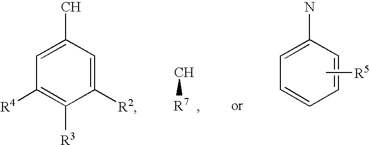
R², R³ and R⁴ are independently hydrogen or a linear or branched alkoxy having from 1 to 4 carbon atoms, wherein when R³ is alkoxy, R² and R⁴ are both hydrogen. R⁵ is hydrogen, linear or branched alkyl having from 1 to 3 carbons, linear or branched alkoxy having from 1 to 3 carbons, or unsubstituted phenoxy. R⁷ is cyclohexyl, cycloheptyl, or a branched alkyl having from 3 to 8 carbon atoms;
R⁸ is alkyl having from 1 to 5 carbon atoms, alkenyl having from 2 to 5 carbon atoms, or alkynyl having from 2 to 5 carbon atoms; and
R⁶ is H or a halogen.

11. The dipeptide of claim 10 wherein X is 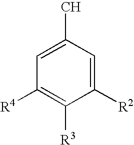
and R³ is alkoxy, and R² and R⁴ are both hydrogen.

12. The dipeptide of claim 10 wherein R³ is OCH₃.

13. The dipeptide of claim 10 wherein R¹ is a branched alkyl group having 4-8 carbon atoms.

14. The dipeptide of claim 13 wherein R¹ is a t-butyl group.

15. The dipeptide of claim 10 wherein R⁸ is an alkyl group.

16. The dipeptide of claim 15 wherein R⁸ is a C4 alkyl group.

17. A method of forming a cyclic melanocortin-4 receptor agonist peptide comprising steps of:
synthesizing an aspartic acid dipeptide of Formula I of claim 10 on a resin;
cleaving the aspartic acid dipeptide from the resin;
providing a second peptide fragment comprising the sequence: D-Phe-Arg-Trp-Lys, wherein the second peptide fragment is attached to a resin;
coupling the carboxyl terminus of the dipeptide to the amino terminus of the second peptide fragment, thereby forming a peptide having sequence [Formula I]-D-Phe-Arg-Trp-Lys;
cyclizing the [Formula I]-D-Phe-Arg-Trp-Lys peptide by covalently coupling the side chain of the aspartic acid residue with the side chain of the lysine residue.


SEQUENCE LISTING (2 sequences)

SEQ ID NO: 1 - protein, 4 aa, Artificial
  Chemically synthesized peptide
FRWK

SEQ ID NO: 2 - protein, 5 aa, Artificial
  Chemically synthesized peptide
DXFRK